(19) United States
(12) Patent Application Publication
(10) Pub. No.: US 2023/0030655 A1
(43) Pub. Date: Feb. 2, 2023

(54) OCCUPATIONAL THERAPY SUPPORT DEVICE, ARTIFICIAL INTELLIGENCE TRAINING DEVICE FOR OCCUPATIONAL THERAPY SUPPORT DEVICE, OCCUPATIONAL THERAPY SUPPORT METHOD, ARTIFICIAL INTELLIGENCE TRAINING METHOD FOR OCCUPATIONAL THERAPY SUPPORT DEVICE, OCCUPATIONAL THERAPY SUPPORT PROGRAM, AND ARTIFICIAL INTELLIGENCE TRAINING PROGRAM

(71) Applicant: Rehabilitation3.0 CO.,LTD., Osaka (JP)

(72) Inventors: Hirokazu MASUDA, Osaka (JP); Shoichi HORIGUCHI, Tokyo (JP); Eiji KUMAKAWA, Tokyo (JP)

(73) Assignee: Rehabilitation3.0 CO.,LTD., Osaka (JP)

(21) Appl. No.: 17/886,131

(22) Filed: Aug. 11, 2022

(30) Foreign Application Priority Data
Jun. 13, 2021 (JP) 2021-098380

(51) Int. Cl. G16H 20/70

(52) U.S. Cl. CPC G16H 20/70

(57) ABSTRACT

An occupational therapy support device disclosed includes: an input data reception unit that receives an input of input data including sleep data of the subject of occupational therapy evaluation and excluding basic data of the subject; an estimation unit that inputs the received input data to a trained artificial intelligence to cause the artificial intelligence to estimate activity of daily living data of the subject and the physical function data of the subject; and an estimated data output unit that outputs the data estimated by the artificial intelligence. The sleep data is data of an item regarding sleep and including at least one of information on pulse, information on respiration, and information on body movements. The activity of daily living data is data of an item regarding activity of daily living and including data evaluated in plural levels on at least one of evaluation items defined in Functional Independence Measure.

### TECHNICAL FIELD

The present invention relates to an occupational therapy support device that supports occupational therapy, a training device that trains an artificial intelligence for the occupational therapy support device, an occupational therapy support method that supports occupational therapy, a training method that trains an artificial intelligence for the occupational therapy support device, an occupational therapy support program that causes a computer to execute an occupational therapy support method, and an artificial intelligence training program that causes a computer to execute an artificial intelligence training method.

### BACKGROUND ART

About 55 years have passed since occupational therapy was legalized in 1965 (Showa 40). During this period, it is said that occupational therapy and occupational therapists have developed and grown as professionals who can contribute to the health condition of the people in various fields such as health, medical, and welfare while responding to changes in social structures and changes in people's awareness of health and disability.

“Occupational therapy” means providing treatment, guidance, and assistance to a person with a physical or mental disorder or a person who is predicted to have a physical or mental disorder, by using work activities that encourage recovery, maintenance, and development of various functions, in order to allow the person to acquire an independent life (definition based on the practice of “occupational therapy” by the Japan Association of Occupational Therapists). The term “occupational” refers to all activities about humans, such as various actions in daily life, work, and play, and means treatment, assistance, or guidance (definition of “occupational” by the Japan Occupational Therapists Association). When performing occupational therapy, a qualified occupational therapist explains first occupational therapy evaluation (assessment) in an interview with the person subjected to prescription and his/her family and performs the occupational therapy evaluation after obtaining consent. In order to evaluate the occupational therapy, the therapist performs information collection from a medical chart and the like, an interview, behavior observation, examination, measurement, and the like. The therapist then prepares an occupational therapy plan and executes the occupational therapy based on the plan. In addition, during therapy, the occupational therapy evaluation is periodically performed, and the effect of the occupational therapy is measured. (For information refer to Non-Patent Literature 1.)

As an effective means for evaluating occupational therapy, functional independence measure (FIM) is known. FIM has been developed in the United States as a method of evaluating how much a person can perform activity of daily living (ADL) by himself/herself. Since the degree of care burden can be particularly evaluated by evaluating activities of daily living by FIM, FIM is a well-known method widely used in the field of rehabilitation and the like also in our country. FIM includes a total of 18 items of movement items and cognitive items, and each item is evaluated in seven levels of 1 point to 7 points. The cognitive items include five items, namely, comprehension, expression, social interaction, problem solving, and memory. The exercise items include 13 items in total, such as eating, grooming, wiping, dressing, going to the bathroom, urination management, defecation management, transfer (sitting-up), and locomotion.

Although FIM is said to be an evaluation method that allows anyone to perform measurement, there is a problem that evaluation results are sometimes inconsistent because of personal judgments by evaluators. When qualified occupational therapists perform evaluation, the variation in evaluation is small. However, in particular, when persons who are not experts in movement perform evaluation, a problem arises. Note that Patent Literature 1 discloses a sleep state determination device that determines a sleep state on the basis of vital data of a user during sleep and subjective data of the user with respect to sleep.

### CITATIONS LIST

### Patent Literature

Patent Literature 1: JP 2019-068907 A

### Non Patent Literature

Non Patent Literature 1: “Occupational Therapy Guidelines (2012 version)”, Japan Association of Occupational Therapists (http://www.jaot.or.jp/wp-content/uploads/2013/08/OTguideline-2012.pdf)

### SUMMARY OF INVENTION

### Technical Problems

The present invention has been made in view of the above problems. One aspect of the present invention has its object to provide an occupational therapy support device that enables a user to obtain data useful for the implementation of occupational therapy and independent of an evaluator. An object of another aspect of the present invention is to provide a training device that trains an artificial intelligence for the occupational therapy support device. An object of still another aspect of the present invention is to provide an occupational therapy support method that enables a user to obtain data useful for the implementation of occupational therapy and independent of an evaluator. An object of still another aspect of the present invention is to provide a training method that trains an artificial intelligence for the occupational therapy support device. An object of still another aspect of the present invention is to provide an occupational therapy support program that causes a computer to execute the occupational therapy support method. An object of still another aspect of the present invention is to provide an artificial intelligence training program that causes a computer to execute an artificial intelligence training method.

### Solutions to Problems

In order to achieve the above object, a device according to the first aspect of the present invention is an occupational therapy support device including an input data reception unit, an estimation unit, and an estimated data output unit. The input data reception unit receives an input of input data including sleep data that is data about the sleep of a subject of occupational therapy and excluding basic data that is data about the body of the subject. The estimation unit inputs the input data whose input has been received by the input data reception unit to a trained artificial intelligence to cause the artificial intelligence to compute the estimated data of data including activity of daily living data that is data about the activity of daily living of the subject. The estimated data output unit outputs the estimated data computed by the artificial intelligence. The sleep data includes data that is based on data measured by a sleep sensor and expressed as a numerical value. The activity of daily living data includes data that is evaluated in plural levels.

According to this configuration, on the basis of sleep data such as the number of turns of the subject of occupational therapy, for example, the estimated data of data regarding activities of daily living such as eating and going to the bathroom, which is useful for occupational therapy evaluation, is obtained. The sleep data, which is input data, is objective data that does not depend on the evaluator. Accordingly, data useful for occupational therapy evaluation that is independent of the evaluator is obtained. In addition, since the input data and the estimated data include data that are expressed as a numerical value or evaluated in plural levels, the estimated data is easily computed by the artificial intelligence. Note that the trained artificial intelligence may be part of the occupational therapy support device having this configuration or may be an external device such as one placed on an external cloud server.

A device according to the second aspect of the present invention is an occupational therapy support device including an input data reception unit, a first estimation unit, a second estimation unit, and an estimated data output unit. The input data reception unit receives an input of input data including sleep data that is data about the sleep of a subject of occupational therapy evaluation and excluding basic data that is data about the body of the subject. The first estimation unit inputs the input data whose input has been received by the input data reception unit to a trained first artificial intelligence to cause the first artificial intelligence to compute the estimated data of data including activity of daily living data that is data about the activity of daily living of the subject. The second estimation unit inputs the estimated data computed by the first artificial intelligence to a trained second artificial intelligence to cause the second artificial intelligence to compute the estimated data of the prescription data of the occupational therapy for the subject. The estimated data output unit outputs the estimated data of the prescription data computed by the second artificial intelligence. The prescription data includes data expressing the contents to be prescribed for at least one item of movement, massage, stretching, and a bedding condition. The sleep data includes data that is based on data measured by a sleep sensor and expressed as a numerical value. The activity of daily living data includes data that is evaluated in plural levels.

According to this configuration, on the basis of sleep data such as the number of turns of the subject of occupational therapy, for example, the estimated data of data regarding activities of daily living such as eating and going to the bathroom, which is useful for occupational therapy evaluation, is computed. The sleep data, which is input data, is objective data that does not depend on the evaluator. Therefore, the computed estimated data is objective data that does not depend on the evaluator. Furthermore, since the estimated data of the prescription data of occupational therapy is obtained based on the computed estimated data, the data can be useful for planning an occupational therapy plan. Since the data that is a basis of the computation is objective data that does not depend on the evaluator, the obtained prescription data is also objective data that does not depend on the evaluator. That is, this configuration provides objective data that does not depend on the evaluator and is useful for making an occupational therapy plan. In addition, since the input data and the estimated data include data that are expressed as a numerical value or evaluated in plural levels, the estimated data is easily computed by the first artificial intelligence. Note that the trained artificial intelligence may be part of the occupational therapy support device having this configuration or may be an external device such as one placed on an external cloud server.

A device according to the third aspect of the present invention is the occupational therapy support device according to the second aspect, in which the prescription data further includes data expressing a movement instruction for at least one item of activity of daily living included in the activity of daily living data.

According to this configuration, it is possible to additionally use a movement instruction regarding at least one item among items regarding the activity of daily living, such as “suppress the water intake” regarding eating, to make an occupational therapy plan. Since the data that is a basis of the computation is objective data that does not depend on the evaluator, the obtained data expressing a movement instruction is also objective data that does not depend on the evaluator.

A device according to the fourth aspect of the present invention is the occupational therapy support device according to any one of the first to third aspects, in which the sleep data is data of an item regarding sleep and including at least one of information on pulse, information on respiration, and information on body movements, and the activity of daily living data is data of an item regarding activity of daily living and including data evaluated in plural levels on at least one of evaluation items defined in Functional Independence Measure.

According to this configuration, since the input data and the estimated data include data that are suited for the computation of the estimated data, the estimated data is computed at a suitable precision. It should be noted that, in this configuration, “at least one of evaluation items defined in Functional Independence Measure” can be “the data evaluated in plural levels” and is not limited to the data evaluated in 7 levels.

A device according to the fifth aspect of the present invention is the occupational therapy support device according to the fourth aspect, in which the sleep data includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

According to this configuration, since the input data includes data that is further suited for the computation of the estimated data, the estimated data is computed at a further suitable precision. It should be noted that, in this configuration, each of various data items from “sleeping time” through “a maximum respiratory rate during the predetermined sleep period—an average respiratory rate during the predetermined sleep period” is for every predetermined time as described as “for every predetermined time.” For example, “a maximum respiratory rate during the predetermined sleep period—an average respiratory rate during the predetermined sleep period” means “a maximum respiratory rate—an average respiratory rate” for “every predetermined time” during “the predetermined sleep period.”

A device according to the sixth aspect of the invention is the occupational therapy support device according to any one of the first to fifth aspects, in which the activity of daily living data is data of an item related to a daily activity including at least one of eating, going to the bathroom, defecation, transfer to the bathroom, locomotion/walk, comprehension, problem solving, and memory.

According to this configuration, the estimated data of the activity of daily living data including a minimum item useful for performing occupational therapy evaluation is computed.

A device according to the seventh aspect of the present invention is the occupational therapy support device according to any one of the first to sixth aspects, in which the data including the activity of daily living data further includes physical function data that is data regarding a physical function of the subject.

According to this configuration, not only the estimated data of the activity of daily living data but also the estimated data of the data regarding physical functions such as grip strength are calculated, and hence it is possible to obtain data useful for occupational therapy evaluation or occupational therapy planning with higher accuracy. Since the sleep data, which is input data, is objective data that does not depend on the evaluator, the estimated data of the physical function data is also objective data that does not depend on the evaluator.

A device according to the eighth aspect of the invention is the occupational therapy support device according to any one of the first to seventh aspects, in which the activity of daily living data includes a fall risk which is a possibility of falling.

According to this configuration, the estimated data of the activity of daily living data including a fall risk is computed, and hence it is possible to obtain data useful for occupational therapy evaluation or occupational therapy planning with higher accuracy.

A device according to the ninth aspect of the present invention is the occupational therapy support device according to any one of the first to eighth aspects, in which the sleep data further includes an answer to a question about sleep from the subject.

According to this configuration, the sleep data includes subjective evaluation data of the subject such as “good sleep”, and hence it is possible to obtain data useful for occupational therapy evaluation or occupational therapy planning with higher accuracy. In addition, since the subjective evaluation data of the subject does not depend on the evaluator, this configuration does not hinder the acquisition of objective data which does not depend on the evaluator and is useful for occupational therapy evaluation or occupational therapy planning.

A device according to the 10th aspect of the present invention is the occupational therapy support device according to any one of the first to ninth aspects, in which the input data further includes environment data that is data regarding an environment of the subject during sleep.

According to this configuration, the input data includes environment data of the subject such as illuminance during sleep, and hence it is possible to obtain data useful for occupational therapy evaluation or occupational therapy planning with higher accuracy. In addition, since the environment data of the subject does not depend on the evaluator, this configuration does not hinder the acquisition of objective data which does not depend on the evaluator and is useful for occupational therapy evaluation or occupational therapy planning.

A device according to the 11th aspect of the present invention is the occupational therapy support device according to any one of the first to 10th aspects, in which the input data includes data including the sleep data of the subject at a plurality of time points from a present to a past in association with time data of the plurality of corresponding time points.

According to this configuration, it is possible to obtain data useful for the occupational therapy evaluation or occupational therapy planning with higher accuracy in consideration of the history of the sleep data and the like (but excluding basic data) of the subject of the occupational therapy evaluation.

A device according to the 12th aspect of the present invention is the occupational therapy support device according to any one of the first to 11th aspects, in which the input data includes the activity of daily living data of the subject at a past time point.

According to this configuration, it is possible to obtain data useful for the occupational therapy evaluation or occupational therapy planning with higher accuracy in consideration of the activity of daily living data of the subject at a past time point.

A device according to the 13th aspect of the present invention is the occupational therapy support device according to the 12th aspect, in which the activity of daily living data of the subject at the past time point is the estimated data estimated at a past by the occupational therapy support device itself.

According to this configuration, it is possible to easily obtain the activity of daily living data at the past time point to be included in the input data.

A device according to the 14th aspect of the present invention is an artificial intelligence training device for an occupational therapy support device that trains the artificial intelligence used by the occupational therapy support device according to the first aspect. The artificial intelligence training device includes an input data reception unit, a training data reception unit, and a training unit. The input data reception unit receives an input of input data including sleep data that is data about the sleep of a subject of occupational therapy evaluation and excluding basic data that is data about the body of the subject. The training data reception unit receives an input of training data that is data corresponding to the input data and including activity of daily living data that is data regarding activity of daily living of the subject. The training unit inputs the input data whose input has been received by the input data reception unit and the training data whose input has been received by the training data reception unit to an artificial intelligence to train the artificial intelligence so as to estimate the training data from the input data. The sleep data includes data that is based on data measured by a sleep sensor and expressed as a numerical value. The activity of daily living data includes data that is evaluated in plural levels.

According to this configuration, the artificial intelligence that can be used for the occupational therapy support device according to the first aspect is constructed by training. Note that the artificial intelligence may be part of the artificial intelligence training device having this configuration or may be an external device such as one placed on an external cloud server.

A device according to the 15th aspect of the present invention is the artificial intelligence training device for the occupational therapy support device according to the 14th aspect, in which the sleep data is data of an item regarding sleep and including at least one of information on pulse, information on respiration, and information on body movements. The activity of daily living data is data of an item regarding activity of daily living and including data evaluated in plural levels on at least one of evaluation items defined in Functional Independence Measure.

According to this configuration, the artificial intelligence that can be used to compute the estimated data of the data including the activity of daily living of the subject in the occupational therapy support device according to the fourth aspect is constructed by training. It should be noted that, in this configuration, “at least one of evaluation items defined in Functional Independence Measure” can be “the data evaluated in plural levels” and is not limited to the data evaluated in 7 levels.

A device according to the 16th aspect of the present invention is the artificial intelligence training device for the occupational therapy support device according to the 15th aspect, in which the sleep data includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

According to this configuration, the artificial intelligence that can be used to compute the estimated data of the data including the activity of daily living of the subject in the occupational therapy support device according to the fifth aspect is constructed by training. It should be noted that, in this configuration, each of various data items from “sleeping time” through “a maximum respiratory rate during the predetermined sleep period—an average respiratory rate during the predetermined sleep period” is for every predetermined time as described as “for every predetermined time.” For example, “a maximum respiratory rate during the predetermined sleep period—an average respiratory rate during the predetermined sleep period” means “a maximum respiratory rate—an average respiratory rate” for “every predetermined time” during “the predetermined sleep period.”

A device according to the 17th aspect of the present invention is the artificial intelligence training device for the occupational therapy support device according to any one of the 14th to 16th aspects, in which the activity of daily living data is data of an item regarding a daily activity including at least one of eating, going to the bathroom, defecation, transfer to the bathroom, locomotion/walk, comprehension, problem solving, and memory.

According to this configuration, the artificial intelligence that can be used to compute the estimated data of the data including the activity of daily living of the subject in the occupational therapy support device according to the sixth aspect is constructed by training.

A device according to the 18th aspect of the present invention is the artificial intelligence training device for an occupational therapy support device according to any one of the 14th to 17th aspects, in which the data including the activity of daily living data further includes physical function data that is data regarding a physical function of the subject.

According to this configuration, an artificial intelligence which can be used to compute the estimated data of data including the activity of daily living data of the subject in the occupational therapy support device according to the seventh aspect is constructed by training.

A device according to the 19th aspect of the invention is the artificial intelligence training device for an occupational therapy support device according to any one of the 14th to 18th aspects, in which the activity of daily living data includes a fall risk which is a possibility of falling.

According to this configuration, an artificial intelligence which can be used to compute the estimated data of data including the activity of daily living data of the subject in the occupational therapy support device according to the eighth aspect is constructed by training.

A device according to 20th aspect of the present invention is the artificial intelligence training device for an occupational therapy support device according to any one of the 14th to 19th aspects, in which the sleep data further includes an answer to a question about sleep from the subject.

According to this configuration, an artificial intelligence which can be used to compute the estimated data of data including the activity of daily living data of the subject in the occupational therapy support device according to the ninth aspect is constructed by training.

A device according to the 21st aspect of the present invention is the artificial intelligence training device for an occupational therapy support device according to any one of the 14th to 20th aspects, in which the input data further includes environment data that is data regarding an environment of the subject during sleep.

According to this configuration, an artificial intelligence which can be used to compute the estimated data of data including the activity of daily living data of the subject in the occupational therapy support device according to the 10th aspect is constructed by training.

A device according to the 22nd aspect of the present invention is the artificial intelligence training device for an occupational therapy support device according to any one of the 14th to 21st aspects, in which the input data includes data including the sleep data of the subject at a plurality of time points from a present to a past in association with time data of the plurality of corresponding time points.

According to this configuration, an artificial intelligence which can be used to compute the estimated data of data including the activity of daily living data of the subject in the occupational therapy support device according to the 11th aspect is constructed by training.

A device according to the 23rd aspect of the present invention is the artificial intelligence training device for an occupational therapy support device according to any one of the 14th to 22nd aspects, in which the input data includes the activity of daily living data of the subject at a past time point.

According to this configuration, an artificial intelligence which can be used to compute the estimated data of data including the activity of daily living data of the subject in the occupational therapy support device according to the 12th aspect is constructed by training.

A device according to the 24th aspect of the present invention is the artificial intelligence training device for an occupational therapy support device according to the 23rd aspect, in which the activity of daily living data of the subject at the past time point is the estimated data estimated at a past by the occupational therapy support device itself.

According to this configuration, an artificial intelligence which can be used to compute the estimated data of data including the activity of daily living data of the subject in the occupational therapy support device according to the 13th aspect is constructed by training.

A device according to the 25th aspect of the present invention is an artificial intelligence training device for an occupational therapy support device that trains the second artificial intelligence used by the occupational therapy support device according to the second aspect. The artificial intelligence training device includes an input data reception unit, a training data reception unit, and a training unit. The input data reception unit receives an input of input data including activity of daily living data that is data regarding activity of daily living of a subject of occupational therapy evaluation. The training data reception unit receives the input of training data which corresponds to the input data and is prescription data of occupational therapy for the subject. The training unit inputs the input data whose input has been received by the input data reception unit and the training data whose input has been received by the training data reception unit to an artificial intelligence that is the second artificial intelligence to train the artificial intelligence so as to estimate the training data from the input data. The prescription data includes data expressing the contents to be prescribed for at least one item of movement, massage, stretching, and a bedding condition. The activity of daily living data includes data that is evaluated in plural levels.

According to this configuration, artificial intelligence that can be used as the second artificial intelligence in the occupational therapy support device according to the second aspect is constructed by training. Note that the artificial intelligence may be part of the artificial intelligence training device having this configuration or may be an external device such as one placed on an external cloud server.

A device according to the 26th aspect of the present invention is the artificial intelligence training device for an occupational therapy support device according to the 25th aspect, in which the activity of daily living data is data of an item regarding activity of daily living and including data evaluated in plural levels on at least one of evaluation items defined in Functional Independence Measure.

According to this configuration, artificial intelligence that can be used as the second artificial intelligence in the occupational therapy support device according to the fourth aspect is constructed by training. It should be noted that, in this configuration, “at least one of evaluation items defined in Functional Independence Measure” can be “the data evaluated in plural levels” and is not limited to the data evaluated in 7 levels.

A device according to the 27th aspect of the present invention is the artificial intelligence training device for an occupational therapy support device according to the 25th or 26th aspect, in which the activity of daily living data is data of an item regarding a daily activity including at least one of eating, going to the bathroom, defecation, transfer to the bathroom, locomotion/walk, comprehension, problem solving, and memory.

According to this configuration, an artificial intelligence that can be used as the second artificial intelligence in the occupational therapy support device according to the sixth aspect is constructed by training.

A device according to the 28th aspect of the present invention is the artificial intelligence training device for the occupational therapy support device according to any one of the 25th to 27th aspects, in which the prescription data further includes data expressing a movement instruction for at least one item of activity of daily living included in the activity of daily living data.

According to this configuration, an artificial intelligence that can be used as the second artificial intelligence in the occupational therapy support device according to the third aspect is constructed by training.

A device according to the 29th aspect of the present invention is the artificial intelligence training device for an occupational therapy support device according to any one of the 25th to 28th aspects, in which the input data further includes physical function data that is data regarding a physical function of the subject.

According to this configuration, an artificial intelligence that can be used as the second artificial intelligence in the occupational therapy support device according to the seventh aspect is constructed by training.

A device according to the 30th aspect of the invention is the artificial intelligence training device for an occupational therapy support device according to any one of the 25th to 29th aspects, wherein the activity of daily living data includes a fall risk which is a possibility of falling.

According to this configuration, an artificial intelligence that can be used as the second artificial intelligence in the occupational therapy support device according to the eighth aspect is constructed by training.

A method according to the 31st aspect of the present invention is an occupational therapy support method, including: (a) receiving, by an occupational therapy support device, the input of input data including sleep data that is data about the sleep of a subject of occupational therapy and excluding basic data that is data about the body of the subject; (b) inputting, by the occupational therapy support device, the input data whose input has been received by the occupational therapy support device to a trained artificial intelligence to cause the artificial intelligence to compute the estimated data of data including activity of daily living data that is data about the activity of daily living of the subject; and (c) outputting, by the occupational therapy support device, the estimated data computed by the artificial intelligence. The sleep data includes data that is based on data measured by a sleep sensor and expressed as a numerical value. The activity of daily living data includes data that is evaluated in plural levels.

The method according to this configuration corresponds to the occupational therapy support method that is implemented by the occupational therapy support device according to the first aspect of the present invention.

A method according to the 32nd aspect of the present invention is an occupational therapy support method, including: (a) receiving, by an occupational therapy support device, an input of input data including sleep data that is data about the sleep of a subject of occupational therapy evaluation and excluding basic data that is data about the body of the subject; (b) inputting, by the occupational therapy support device, the input data whose input has been received by the occupational therapy support device to a trained first artificial intelligence to cause the first artificial intelligence to compute the estimated data of data including activity of daily living data that is data about the activity of daily living of the subject; (c) inputting, by the occupational therapy support device, the estimated data computed by the first artificial intelligence to a trained second artificial intelligence to cause the second artificial intelligence to compute the estimated data of the prescription data of the occupational therapy for the subject; and (d) outputting, by the occupational therapy support device, the estimated data of the prescription data computed by the second artificial intelligence. The prescription data includes data expressing the contents to be prescribed for at least one item of movement, massage, stretching, and a bedding condition. The sleep data includes data that is based on data measured by a sleep sensor and expressed as a numerical value. The activity of daily living data includes data that is evaluated in plural levels.

The method according to this configuration corresponds to the occupational therapy support method that is implemented by the occupational therapy support device according to the second aspect of the present invention.

A method according to the 33rd aspect of the present invention is the occupational therapy support method according to the 32nd aspect, in which the prescription data further includes data expressing a movement instruction for at least one item of activity of daily living included in the activity of daily living data.

The method according to this configuration corresponds to the occupational therapy support method that is implemented by the occupational therapy support device according to the third aspect of the present invention.

A method according to the 34th aspect of the present invention is the occupational therapy support method according to any one of the 31st to 33rd aspects, in which the sleep data is data of an item regarding sleep and including at least one of information on pulse, information on respiration, and information on body movements, and the activity of daily living data is data of an item regarding activity of daily living and including data evaluated in plural levels on at least one of evaluation items defined in Functional Independence Measure.

The method according to this configuration corresponds to the occupational therapy support method that is implemented by the occupational therapy support device according to the fourth aspect of the present invention. It should be noted that, in this configuration, “at least one of evaluation items defined in Functional Independence Measure” can be “the data evaluated in plural levels” and is not limited to the data evaluated in 7 levels.

A method according to the 35th aspect of the present invention is the occupational therapy support method according to the 34th aspect, in which the sleep data includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

The method according to this configuration corresponds to the occupational therapy support method that is implemented by the occupational therapy support device according to the fifth aspect of the present invention. It should be noted that, in this configuration, each of various data items from “sleeping time” through “a maximum respiratory rate during the predetermined sleep period—an average respiratory rate during the predetermined sleep period” is for every predetermined time as described as “for every predetermined time.” For example, “a maximum respiratory rate during the predetermined sleep period—an average respiratory rate during the predetermined sleep period” means “a maximum respiratory rate—an average respiratory rate” for “every predetermined time” during “the predetermined sleep period.”

A method according to the 36th aspect of the present invention is the occupational therapy support method according to any one of the 31st to 35th aspects, in which the input data includes data including the sleep data of the subject at a plurality of time points from a present to a past in association with time data of the plurality of corresponding time points.

The method according to this configuration corresponds to the occupational therapy support method that is implemented by the occupational therapy support device according to the 11th aspect of the present invention.

A method according to the 37th aspect of the present invention is an artificial intelligence training method for an occupational therapy support device that trains the artificial intelligence used by the occupational therapy support device according to the first aspect. The artificial intelligence training method includes: (a) receiving, by the artificial intelligence training device, an input of input data including sleep data that is data about the sleep of a subject of occupational therapy evaluation and excluding basic data that is data about the body of the subject; (b) receiving, by the artificial intelligence training device, an input of training data that is data corresponding to the input data and including activity of daily living data that is data regarding activity of daily living of the subject; and (c) inputting, by the artificial intelligence training device, the input data whose input has been received by the artificial intelligence training device and the training data whose input has been received by the artificial intelligence training device to the artificial intelligence to train the artificial intelligence so as to estimate the training data from the input data. The sleep data includes data that is based on data measured by a sleep sensor and expressed as a numerical value. The activity of daily living data includes data that is evaluated in plural levels.

The method according to this configuration corresponds to the artificial intelligence training method that is implemented by the artificial intelligence training device for the occupational therapy support device according to the 14th aspect of the present invention.

A method according to the 38th aspect of the present invention is the artificial intelligence training method for the occupational therapy support device according to the 37th aspect, in which the sleep data is data of an item regarding sleep and including at least one of information on pulse, information on respiration, and information on body movements. The activity of daily living data is data of an item regarding activity of daily living and including data evaluated in plural levels on at least one of evaluation items defined in Functional Independence Measure.

The method according to this configuration corresponds to the artificial intelligence training method that is implemented by the artificial intelligence training device for the occupational therapy support device according to the 15th aspect of the present invention. It should be noted that, in this configuration, “at least one of evaluation items defined in Functional Independence Measure” can be “the data evaluated in plural levels” and is not limited to the data evaluated in 7 levels.

A method according to the 39th aspect of the present invention is the artificial intelligence training method for the occupational therapy support device according to the 38th aspect, in which the sleep data includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

The method according to this configuration corresponds to the artificial intelligence training method that is implemented by the artificial intelligence training device for the occupational therapy support device according to the 16th aspect of the present invention. It should be noted that, in this configuration, each of various data items from “sleeping time” through “a maximum respiratory rate during the predetermined sleep period—an average respiratory rate during the predetermined sleep period” is for every predetermined time as described as “for every predetermined time.” For example, “a maximum respiratory rate during the predetermined sleep period—an average respiratory rate during the predetermined sleep period” means “a maximum respiratory rate—an average respiratory rate” for “every predetermined time” during “the predetermined sleep period.”

A method according to the 40th aspect of the present invention is the artificial intelligence training method for the occupational therapy support device according to any one of the 37th to 39th aspects, in which the input data includes data including the sleep data of the subject at a plurality of time points from a present to a past in association with time data of the plurality of corresponding time points.

The method according to this configuration corresponds to the artificial intelligence training method that is implemented by the artificial intelligence training device for the occupational therapy support device according to the 22nd aspect of the present invention.

A method according to the 41st aspect of the present invention is an artificial intelligence training method for an occupational therapy support device that trains the second artificial intelligence used by the occupational therapy support device according the second aspect. The artificial intelligence training method includes: (a) receiving, by the artificial intelligence training device, an input of input data including activity of daily living data that is data regarding activity of daily living of the subject of occupational therapy evaluation; (b) receiving, by the artificial intelligence training device, an input of training data which corresponds to the input data and is prescription data of occupational therapy for the subject; and (c) inputting, by the artificial intelligence training device, the input data whose input has been received by the artificial intelligence training device and the training data whose input has been received by the artificial intelligence training device, to an artificial intelligence that is the second artificial intelligence to train the artificial intelligence so as to estimate the training data from the input data. The prescription data includes data expressing the contents to be prescribed for at least one item of movement, massage, stretching, and a bedding condition. The activity of daily living data includes data that is evaluated in plural levels.

The method according to this configuration corresponds to the artificial intelligence training method that is implemented by the artificial intelligence training device for the occupational therapy support device according to the 25th aspect of the present invention.

A method according to the 42nd aspect of the present invention is the artificial intelligence training method for the occupational therapy support device according to the 41st aspect, in which the activity of daily living data is data of an item regarding activity of daily living and including data evaluated in plural levels on at least one of evaluation items defined in Functional Independence Measure.

The method according to this configuration corresponds to the artificial intelligence training method that is implemented by the artificial intelligence training device for the occupational therapy support device according to the 26th aspect of the present invention. It should be noted that, in this configuration, “at least one of evaluation items defined in Functional Independence Measure” can be “the data evaluated in plural levels” and is not limited to the data evaluated in 7 levels.

A method according to the 43rd aspect of the present invention is the artificial intelligence training method for the occupational therapy support device according to the 41st or 42nd aspect, in which the prescription data further includes data expressing a movement instruction for at least one item of activity of daily living included in the activity of daily living data.

The method according to this configuration corresponds to the artificial intelligence training method that is implemented by the artificial intelligence training device for the occupational therapy support device according to the 28th aspect of the present invention.

A method according to the 44th aspect of the present invention is an occupational therapy support program, the program, by being read by a computer, causing the computer to execute the occupational therapy support method according to any of the 31st to 36th aspects as the occupational therapy support device.

According to this configuration, the occupational therapy support method according to any one of the 31st to 36th aspects is implemented by a computer.

A method according to the 45th aspect of the present invention is an artificial intelligence training program, the program, by being read by a computer, causing the computer to execute the artificial intelligence training method according to any one of the 37th to 43rd aspects as the artificial intelligence training device.

According to this configuration, the artificial intelligence training method according to any one of the 37th to 43rd aspects is implemented by a computer.

### Advantageous Effects of Invention

As described above, one aspect of the present invention implement an occupational therapy support device, an occupational therapy support method, or an occupational therapy support program that enables a user to obtain data useful for the implementation of occupational therapy and independent of an evaluator. In addition, another aspect of the present invention implements an artificial intelligence training device, an artificial intelligence training method, or an artificial intelligence training program that trains an artificial intelligence for the occupational therapy support device.

Objects, features, aspects, and advantages of the present invention will become more apparent from the following detailed description and the accompanying drawings.

### BRIEF DESCRIPTION OF DRAWINGS

FIG. 1 is a diagram illustrating the configuration of an occupational therapy support system including an occupational therapy support device according to an embodiment of the present invention.

FIG. 2 is a block diagram illustrating the configuration of the occupational therapy support device in FIG. 1.

FIGS. 3A to 3D are tabular diagrams illustrating input data and output data of the occupational therapy support device illustrated in FIG. 2.

FIG. 4 is a schematic diagram illustrating the conceptual configuration of an artificial intelligence of the occupational therapy support device illustrated in FIG. 2.

FIG. 5 is a block diagram illustrating the configuration of an occupational therapy support device according to another embodiment of the invention.

FIG. 6 is a tabular diagram illustrating output data of the occupational therapy support device illustrated in FIG. 5.

FIG. 7 is a block diagram illustrating the configuration of an occupational therapy support device according to still another embodiment of the invention.

FIG. 8 is a block diagram showing input data and output data used in a verification test of the occupational therapy support device illustrated in FIG. 7.

FIGS. 9A to 9C are explanatory diagrams showing an evaluation method used in the verification test of the occupational therapy support device illustrated in FIG. 7.

FIG. 10 is a graph showing the results of the verification test of the occupational therapy support device illustrated in FIG. 7.

FIG. 11 is a graph showing the results of the verification test of the occupational therapy support device illustrated in FIG. 7.

FIG. 12 is a flowchart illustrating a processing flow of the occupational therapy support method implemented by the occupational therapy support device illustrated in FIG. 2 or FIG. 7.

FIG. 13 is a flowchart illustrating a processing flow of the occupational therapy support method implemented by the occupational therapy support device illustrated in FIG. 5.

FIG. 14 is a flowchart illustrating a processing flow of the artificial intelligence training method implemented by the occupational therapy support device illustrated in FIG. 2, FIG. 5, or FIG. 7.

### DESCRIPTION OF EMBODIMENTS

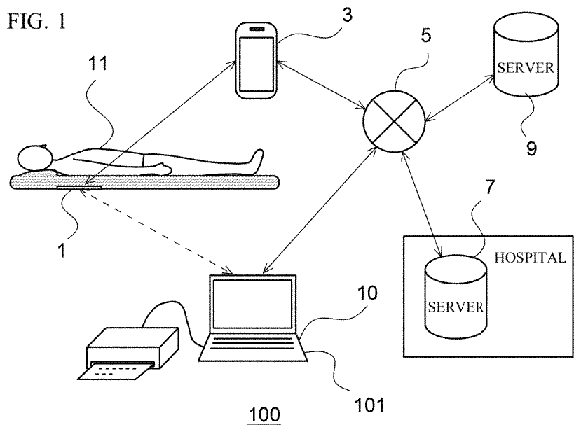

FIG. 1 is a diagram illustrating the configuration of an occupational therapy support system including an occupational therapy support device according to an embodiment of the present invention. An occupational therapy support system 100 includes a sleep sensor 1, a user communication terminal 3, a network 5, and servers 7 and 9 in addition to an occupational therapy support device 101. The sleep sensor 1, the user communication terminal 3, the network 5, and the servers 7 and 9 are devices that are directly or indirectly connected to the occupational therapy support device 101 and cooperate with the occupational therapy support device 101.

The occupational therapy support device 101 is a device that supports occupational therapy by outputting activity of daily living data and the estimated data of physical function data for performing occupational therapy evaluation based on the sleep data and the basic data of a subject 11 of the occupational therapy evaluation. The occupational therapy support device 101 is incorporated in the computer 10 in the illustrated example. That is, installing and activating a specific application in the computer 10 causes a processing device (processor) such as a central processing unit (CPU) of the computer 10 to function as the occupational therapy support device 101.

The sleep sensor 1 is a sensor that automatically acquires the sleep data of the subject 11 of occupational therapy evaluation (hereinafter abbreviated as “subject”) and has a communication function of transmitting the acquired data to the user communication terminal 3 or the occupational therapy support device 101 by radio or the like. The sleep data is data about the sleep of the subject 11, such as the sleeping time, the number of turns, respiration during sleep, and pulse. In the illustrated example, the sleep sensor 1 is a mat-shaped sensor that is used while being placed under the bedding on which the subject 11 lies. The sleep sensor 1 in this form is also already commercially available and well known. As the sleep sensor 1, a sleep sensor that can simultaneously collect environment data during sleep, such as a room temperature, humidity, and illuminance is also known. Each item of sleep data and environment data will be described later.

In the illustrated example, the user communication terminal 3 is a smartphone owned by the user. The user is, for example, the subject 11 himself/herself or a relative who takes care of the subject 11. Installing a specific application in advance in the user communication terminal 3 allows the user communication terminal 3 to communicate with the sleep sensor 1. In communication, it is possible to prevent information leakage by, for example, requiring the input of an identification code (ID) and a password. The measurement data received by the user communication terminal 3 from the sleep sensor 1 is transmitted to the occupational therapy support device 101 via the network 5. Even in communication between the user communication terminal 3 and the occupational therapy support device 101, it is possible to prevent information leakage by, for example, requiring the input of an identification code (ID) and a password. The user can also input, to the user communication terminal 3, subjective evaluation data regarding the sleep of the subject 11, for example, a selection result regarding sleepiness namely “had a good sleep”, “could not say either”, or “did not sleep”. The user can also photograph the bedding state of the subject 11 and transmit the bedding state as one of environment data to the occupational therapy support device 101 using the user communication terminal 3. For example, when an application installed in the user communication terminal 3 is activated, a question or an instruction is displayed, and the subjective evaluation data and the environment data can be input or photographed in a format responding to the question or instruction.

The network 5 is the Internet in the illustrated example. The server 7 is a server that is held by a facility such as a hospital and holds basic data such as a medical record of the subject 11 and is connected to the network 5. The server 7 may be a server that is held by an external agency and used by a facility such as a hospital. The occupational therapy support device 101 can acquire basic data such as the age, medical history, and the like of the subject 11 by accessing the server 7. Even in communication between the occupational therapy support device 101 and the server 7, it is possible to prevent information leakage by, for example, requiring the input of an identification code (ID) and a password. Each item of the basic data will also be described later.

The server 9 is connected to the network 5 and constructs an artificial intelligence which can be used via the network 5. The occupational therapy support device 101 estimates activity of daily living data and physical function data based on the sleep data and basic data of the subject 11 of occupational therapy evaluation using the artificial intelligence. The activity of daily living data is data of items about daily activities, such as eating, going to the bathroom, defecation, transfer to the bathroom, locomotion/walk, comprehension, problem solving, and memory. The physical function data is data of items about physical functions such as grip strength. Each item of daily life data and physical function data will also be described later. The artificial intelligence may be constructed in the computer 10 as a part of the occupational therapy support device 101, may be constructed in the computer 10 so that the occupational therapy support device 101 can access the artificial intelligence separately from the occupational therapy support device 101, or may be an artificial intelligence outside the computer 10 like the artificial intelligence provided by the server 9. Even in communication between the occupational therapy support device 101 and the server 9, it is possible to prevent information leakage by, for example, requiring the input of an identification code (ID) and a password.

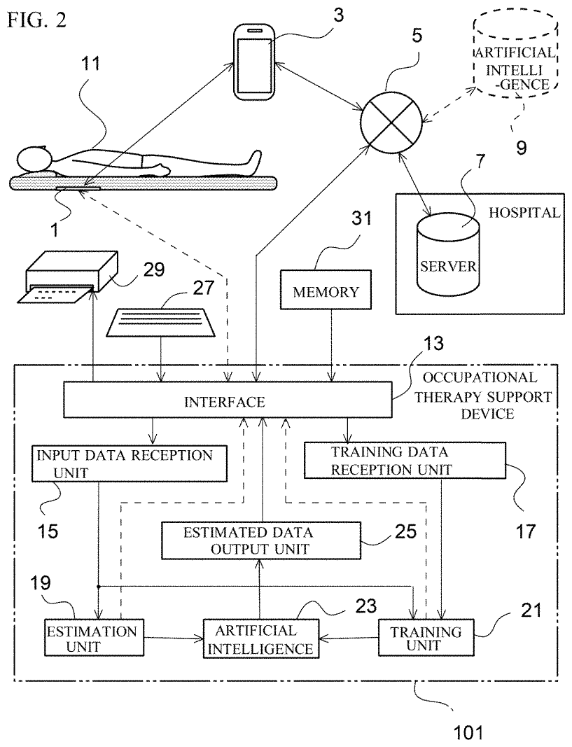

FIG. 2 is a block diagram illustrating the configuration of the occupational therapy support device 101. The occupational therapy support device 101 includes an interface 13, an input data reception unit 15, a training data reception unit 17, an estimation unit 19, a training unit 21, an artificial intelligence 23, and an estimated data output unit 25. The interface 13 is a device portion that enables communication between the occupational therapy support device 101 itself and an external device in accordance with a predetermined protocol for each external device. Communication between the sleep sensor 1, the user communication terminal 3, the servers 7 and 9, an input device 27 such as a keyboard, an output device 29 such as a printer or display, a storage medium 31 such as a USB memory or CDROM, and the occupational therapy support device 101 is performed via the interface 13.

The input data reception unit 15 receives the input of input data including the sleep data of the subject 11 and the basic data of the subject 11. The estimation unit 19 inputs the input data whose input has been received by the input data reception unit 15 to the artificial intelligence 23 to cause the artificial intelligence 23 to compute estimated data regarding the activity of daily living data and the physical function data of the subject 11. If training has already been performed, the artificial intelligence 23 outputs the estimated data with high accuracy concerning the activity of daily living data and the physical function data. The estimated data output unit 25 outputs the estimated data computed by the artificial intelligence 23. The estimated data output by the estimated data output unit 25 is transmitted to, for example, the user communication terminal 3 or the output device 29 via the interface 13. This allows the occupational therapist or the user to obtain the estimated data of the items of occupational therapy evaluation. The occupational therapist who has received the estimated data directly or via the user can prepare an occupational therapy plan based on the received estimated data and perform occupational therapy based on the prepared plan.

The computer 10 (see FIG. 1) in which the occupational therapy support device 101 is incorporated may be provided in, for example, a facility of an occupational therapist, a hospital, or the like, or may be a mobile computer that can be carried by the occupational therapist to the home of the subject 11. When the subject 11 is hospitalized in a facility, a hospital, or the like and the computer 10 is equipment in the facility, the hospital, or the like, it is possible to directly perform communication between the occupational therapy support device 101 and the sleep sensor 1 without via the user communication terminal 3.

The artificial intelligence 23 can output estimated data with high accuracy through machine learning. The occupational therapy support device 101, since including the training data reception unit 17 and the training unit 21, can train the artificial intelligence 23 by itself without using an external artificial intelligence training device. That is, the occupational therapy support device 101 also incorporates an artificial intelligence training device that trains the artificial intelligence 23 as machine learning. When the occupational therapy support device 101 performs machine learning, the input data reception unit 15 receives the input of input data including sleep data and basic data, and the training data reception unit 17 receives the input of training data that is a set of correct activity of daily living data and physical function data corresponding to these input data. The training unit 21 inputs the input data whose input has been received by the input data reception unit 15 and the training data received by the training data reception unit 17 to the artificial intelligence 23 to train the artificial intelligence 23 so as to estimate the training data from the input data. Inputting a large number of sets of input data and training data associated with each other to the occupational therapy support device 101 will foster the training of the artificial intelligence 23 and improve the estimation accuracy.

In the past, the sleep data and the basic data collected for various subjects 11 and the activity of daily living data and the physical function data obtained by actual measurement in correspondence with these data are recorded in the storage medium 31 in association with each other, for example, so that the input data reception unit 15 and the training data reception unit 17 sequentially read a large number of data required for training from the storage medium 31, and the training unit 21 can repeat the training of the artificial intelligence 23 for each read data. As described above, the occupational therapy support device 101 can switch and execute two operation modes, namely, an estimation mode for computing and outputting estimated data using the artificial intelligence 23 and a training mode for training the artificial intelligence 23 as machine learning. For example, the input device 27 can instruct the switching of the operation modes.

In the example of FIG. 2, the artificial intelligence 23 is incorporated in the computer 10 as part of the occupational therapy support device 101. On the other hand, as illustrated by the dotted line in FIG. 2, the artificial intelligence constructed in the external server 9 or the like may be used. In this case, the estimation unit 19, the training unit 21, and the estimated data output unit 25 operate the external artificial intelligence via the network 5 and the like. The estimated data output unit 25 causes the external artificial intelligence to output estimated data, for example, receives the estimated data by the estimated data output unit 25 via the interface 13, and further outputs the received estimated data to the output device 29, the user communication terminal 3, or the like via the interface 13 by the estimated data output unit 25. When the external artificial intelligence is used, the artificial intelligence 23 constituting a part of the occupational therapy support device 101 is unnecessary.

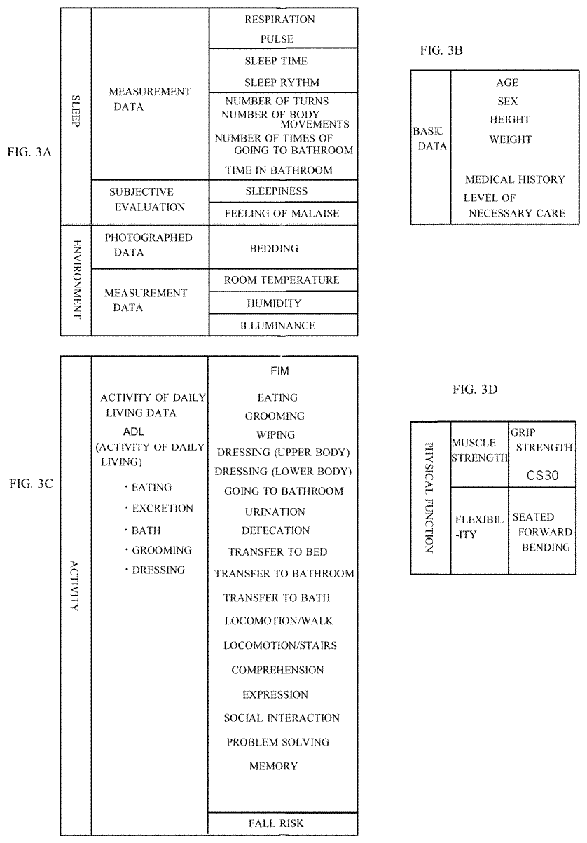

FIGS. 3A to 3D are tabular diagrams illustrating input data and output data of the occupational therapy support device 101. FIG. 3A illustrates sleep data and environment data during sleep, FIG. 3B illustrates basic data, FIG. 3C illustrates activity of daily living data, and FIG. 3D illustrates physical function data. The following will exemplify how to express each data to be handled by the occupational therapy support device 101. Obviously, this is merely an example and other expressions can be used.

Of the sleep data (see FIG. 3A), the sleep time and the time in the bathroom are expressed in units of time (h) as 6.5. The sleep rhythm is represented by a time-series change in sleep time and wakefulness time and is represented by, for example, a data string such as (WAKEFULNESS, WAKEFULNESS, SLEEP, SLEEP, SLEEP, SLEEP, WAKEFULNESS, WAKEFULNESS, SLEEP, . . . ) indicating whether the sleep state or the wakefulness state is observed at 15 minute intervals from the time of getting into bed to 9 hours later. This makes it possible to obtain the time from lying down to falling asleep, which is an index of good or bad falling asleep. “Sleep” and “wakefulness” are represented by codes assigned in advance, for example, numerical values “1” and “0”. The number of turns, the number of body movements, and the number of times of going to the bathroom are represented by natural numbers such as 1, 2, and 3. The number of body movements is the number of movements performed during lying down, excluding turning over, and means, for example, the number of movements such as moving a foot or taking out a hand from a comforter. The number of times of going to the bathroom means the number of times of leaving the bed for going to the bathroom during the sleeping time. Respiration and pulse each are represented by the number of times within one minute. A room temperature, humidity, and illuminance, which are environment data, are represented by numerical values based on the units of temperature, humidity, and illuminance, respectively. The sleep data and the environment data are acquired by the sleep sensor 1. Alternatively, the above sleep data may be generated by the application of the user communication terminal 3 or the application of the computer 10 on the basis of the raw data acquired by the sleep sensor 1. That is, data regarding, for example, lying down, sleep, wakefulness, turning over, and body movement is generated by the sleep sensor 1 itself or the application from a change in pressure, a heart rate, a respiratory rate, and the like sensed by the sleep sensor 1. Even when it is not possible to specify which part of the body has been moved with regard to the body movement, it is possible to detect that the body movement is not rolling movement.

Of the subjective evaluation data included in the sleep data, the sleepiness includes any one of the options namely “had a good sleep”, “could not say either”, and “did not sleep”, and each option is represented by a code assigned in advance, for example, numerical values “1”, “2”, and “3”. Alternatively, each option may be represented by a code corresponding to selection or non-selection, for example, a numerical value such as “1” or “0”. The feeling of malaise includes, for example, “pleasant”, “average”, and “dull”. These subjective evaluation data are input, for example, by a user, an occupational therapist, or the like touching an option displayed on the screen of the user communication terminal 3 or the computer 10 in which the application is activated. For example, dialog “Sleep well?” appears on the screen as a question about sleepiness, and “I had a good sleep”, “I cannot say either”, and “I did not sleep” are simultaneously displayed as options. When the user or the like touches the option “I had a good sleep”, a code corresponding to the option “I had a good sleep” is input to the application. This code is input to the occupational therapy support device 101 as an answer to sleepiness.

Of the environment data, the bedding is photographic data of the bedding. The photograph data is photographed by the camera attached to the user communication terminal 3 or the computer 10 in which the application is activated. The image data acquired by photographing is input to the occupational therapy support device 101. The image data is represented by a set of pixel values.

The basic data (see FIG. 3B) is acquired from, for example, the server 7 of the hospital. Alternatively, for example, the information may be manually input to the user communication terminal 3 or the computer 10 that has started the application. Of the basic data, the age, height, and weight are represented by numerical values based on these units. The sex is represented by a code corresponding to a male or a female, for example, a numerical value such as “0” or “1”. The medical history is represented by codes assigned in advance to various disease names, for example, numerical values such as “0”, “1”, “2”, Alternatively, each disease name may be expressed by a reference sign corresponding to “absent” or “present”, for example, a numerical value such as “0” or “1”. The degree of care represents the level of necessary care, and is represented by, for example, numerical values in eight levels.

The activity of daily living data (see FIG. 3C) includes 18 items based on the functional independence rating method (FIM), which is known as an effective means of occupational therapy evaluation. For the daily movement of each item, how much the subject 11 can perform by himself/herself is evaluated in seven levels of 1 point to 7 points. The activity of daily living data includes cognitive items and movement items. The cognitive items include five items namely comprehension, expression, social interaction, problem solving, and memory. The movement items include other 13 items namely eating, grooming, wiping, dressing, going to the bathroom, urination management, defecation management, transfer (sitting-up movement), and locomotion. Each item is expressed by a numerical value corresponding to a score. The activity of daily living data further includes “fall risk”. A fall risk is obtained by evaluating the possibility of falling and is expressed by, for example, numerical values in two levels namely “0” and “1” corresponding to “high possibility” and low possibility”, respectively, or numerical values such as “0”, “1”, “2”, and “3” corresponding to many levels further subdivided.

Of the physical function data (see FIG. 3D), the grip strength as an index of muscle strength is represented by a numerical value (for example, a numerical value in kgw) representing the grip strength. CS30, which is also known as an index of muscle strength, represents how many times a person can stand up from a chair in 30 seconds and is represented by a numerical value indicating the number of times. Of the physical function data, seated forward bending as an index of flexibility represents how much the position of the hands moves forward when the body is bent forward while the arms are extended forward in a long sitting posture and is expressed, for example, in units of centimeters. The estimated data output unit 25 converts the estimated values computed by the artificial intelligence 23, regarding the activity of daily living data expressed by discontinuous numerical values in seven levels, into numerical values each closest to one of the numerical values in seven levels by, for example, rounding off and outputs the converted values. Since the data after the conversion is based on the estimated data of the activity of daily living data computed by the artificial intelligence 23, the data after the conversion is still the estimated data of the activity of daily living data.

In order to solve the problem of obtaining an objective occupational therapy evaluation that does not depend on the evaluator, the inventor of the present application having long-time experience as an occupational therapist has conceived to estimate activity of daily living data and physical function data on the basis of sleep data and basic data that are objective data that do not depend on an evaluator. It was expected that there was a complex but correlated relationship between a set of sleep data and basic data and a set of activity of daily living data and physical function data. Therefore, the present inventor has considered that it would be possible in principle to estimate activity of daily living data and physical function data on the basis of the sleep data and the basic data even if it is an excessive burden and is not realistic to perform such estimation by human intelligence. Then, it has been conceived that acquisition of such estimated data of occupational therapy evaluation beyond human intelligence can be made realistic by using an artificial intelligence. Since sleep data and basic data as the foundation are objective data that do not depend on the evaluator, the obtained activity of daily living data and the estimated data of physical function data are also objective data that do not depend on the evaluator.

On the basis of the experience of the occupational therapist, it is sufficient to have data of items namely eating, going to the bathroom, defecation, transfer to the bathroom, locomotion/walk, comprehension, problem solving, and memory as activity of daily living data in order to perform occupational therapy evaluation as a premise for drafting an occupational therapy plan. If there is data of an item of grip strength as physical function data and there are data of more items, more accurate occupational therapy evaluation can be obtained. Similarly, based on the experience of the occupational therapist, in order to obtain the minimum items of the above-described occupational therapy evaluation, data of items namely the number of turns, the number of body movements, the number of times of going to the bathroom, and the time in the bathroom is sufficient as sleep data, and data of the items namely age, height, weight, and medical history is sufficient as basic data. Conversely, even when the number of items of activity of daily living data, sleep data, and basic data is smaller than the number of items described above, the obtained estimated data can be useful for occupational therapy evaluation. Obtained estimated data is objective data that does not depend on the evaluator as long as it is based on objective data that does not depend on the evaluator, regardless of whether the number of items is large or small.

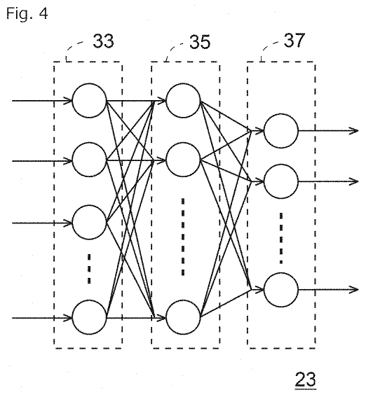

FIG. 4 is a schematic diagram illustrating the conceptual configuration of the artificial intelligence 23 used by the occupational therapy support device 101. The artificial intelligence provided by the server 9 has a similar configuration as an example. The illustrated artificial intelligence 23 is a neural network and includes an input layer 33 in which nodes receiving the input of data are arranged, an output layer 37 in which nodes outputting operation result data are arranged, and an intermediate layer 35 in which nodes connecting the input layer 33 and the output layer 37 are arranged. In the illustrated example, the intermediate layer 35 is single, but may span multiple layers. The value of the previous node is transmitted to the next node while reflecting the parameter given to each node, that is, the weight and the bias value of each node. The input layer 33 receives the input data whose input has been received by the input data reception unit 15, that is, a set of items of sleep data and basic data. The input data is transmitted to the output layer 37 via the intermediate layer 35 while reflecting the parameter of each node. The data transmitted to the output layer 37 is the estimated data of a set of items of the activity of daily living data and the physical function data. The estimation unit 19 (see FIG. 2) inputs a set of items of the sleep data and the basic data of the subject 11 to the input layer 33 of the artificial intelligence 23 and causes the output layer 37 to generate the activity of daily living data and the estimated data of the physical function data of the subject 11. The estimated data output unit 25 outputs the generated estimated data after performing conversion such as rounding off or without performing conversion.

In order for estimated data appearing at a node of the output layer 37 to be the estimated data of the activity of daily living data and the physical function data with high accuracy, it is necessary to train the artificial intelligence 23 using the actually measured activity of daily living data and physical function data. Training is performed by inputting, to the input layer 33, a set of items of the sleep data and the basic data of a certain subject 11 whose input has been received by the input data reception unit 15, and inputting, to the output layer 37, training data about the same subject 11 received by the training data reception unit 17, that is, a set of items of the measured activity of daily living data and the physical function data as training data. The training unit 21 (see FIG. 2) inputs such data to the artificial intelligence 23.

The artificial intelligence 23 computes the estimated data of activity of daily living data and physical function data based on the input sleep data and the basic data, generates the estimated data in the output layer 37, and computes an error between the generated estimated data and the activity of daily living data and the physical function data input as training data. The artificial intelligence 23 then changes the parameter of each node from the output layer 37 toward the input layer 33 by, for example, a well-known error back propagation algorithm so as to generate estimated data without error. Such a function is included in the artificial intelligence 23 itself. Preparing a large number of sets of input data and training data and repeating training will cause the artificial intelligence 23 to compute estimated data with high accuracy. When the artificial intelligence 23 is trained, the number of intermediate layers 35 and the number of nodes of each layer can be adjusted to optimum values. Such techniques are also well known.

In order to obtain the estimated data of the activity of daily living data and the physical function data of the subject 11, it is possible to input not only the latest data to the artificial intelligence 23, concerning the sleep data and the basic data of the subject 11, but also input data at a plurality of time points including data before the latest data to the artificial intelligence 23 together with the time data of each data. As a result, regarding the sleep data and the basic data of the subject 11, the estimated data of the activity of daily living data and the physical function data of the same subject 11 are obtained in consideration of also the past history. This makes it possible to obtain estimated data with higher accuracy. Time data may be represented by, for example, the date and time at each time point or may be represented by a date and time difference from the latest time point. The input data reception unit 15 receives data at a plurality of time points together with the respective time data, and the estimation unit 19 inputs the data at the plurality of time points and the respective time data to the input layer 33 of the artificial intelligence 23. The larger the number of time points of the input data, the more the number of nodes of the input layer 33 receiving the input of the data increases in proportion thereto.

In order to obtain estimated data based on data at a plurality of time points, it is necessary to train the artificial intelligence 23 based on the data at the plurality of time points, the respective time data, and the training data corresponding to the respective data. For example, in order to obtain the estimated data of activity of daily living data and physical function data from sleep data and basic data at past three time points including the latest time point, the artificial intelligence 23 can be trained by inputting the sleep data and the basic data at the three time points of various subjects 11 and the respective time data to the input layer 33 and inputting the latest actual measurement data of the activity of daily living data and the physical function data of each subject 11 to the output layer 37. Since the time data is simultaneously input, a plurality of time points at which sleep data and basic data are collected may be different among different subjects 11. For example, data at the latest time point, 1 week ago, and 5 weeks ago may be input for a certain subject 11, and data at the latest time point, 3 weeks ago, and 15 weeks ago may be input for another subject. The artificial intelligence 23 adjusts the parameter of the node so as to compute estimated data reflecting the influence of the temporal distance from the latest time point through training with a large number of data.

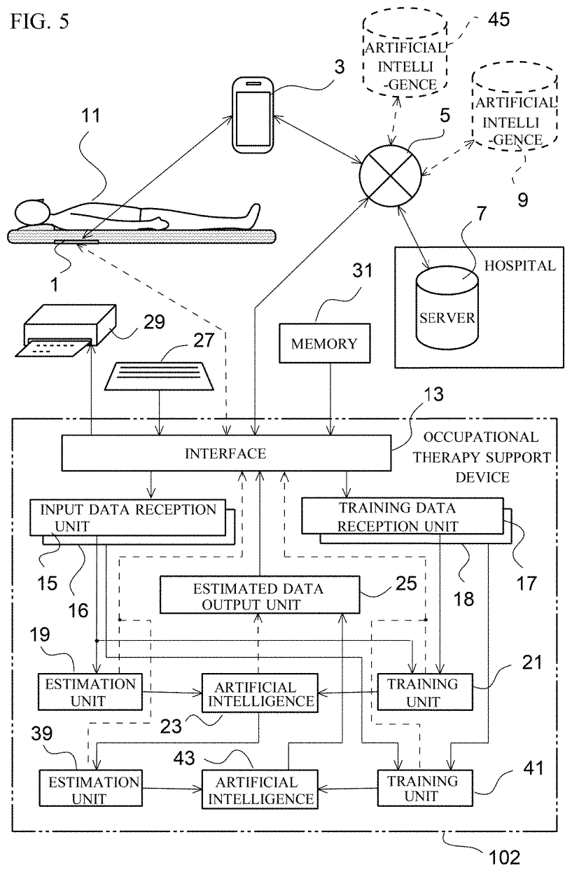

FIG. 5 is a block diagram illustrating the configuration of an occupational therapy support device according to another embodiment of the invention. An occupational therapy support device 102 is different from the occupational therapy support device 101 (see FIG. 2) in further including another input data reception unit 16, another training data reception unit 18, another estimation unit 39, another artificial intelligence 43, and another training unit 41. In the occupational therapy support device 102, the estimation unit 39 reads out the estimated data computed by the artificial intelligence 23, that is, the estimated data regarding the activity of daily living data and the physical function data of the subject 11. The estimation unit 39 inputs the read estimated data of the artificial intelligence 23 to the artificial intelligence 43, thereby causing the artificial intelligence 43 to compute the estimated data regarding the prescription data expressing the information to be prescribed to the subject 11. Similar to the estimated data output unit 25, the estimation unit 39 inputs the read estimated data of the artificial intelligence 23 to the artificial intelligence 43 upon converting the estimated data by rounding off or the like or without conversion.

If training has already been performed, the artificial intelligence 43 outputs estimated data with high accuracy regarding prescription data. The estimated data output unit 25 converts the estimated data computed by the artificial intelligence 43 or outputs the estimated data without conversion. The estimated data output by an estimated data output unit 45 is transmitted to, for example, the user communication terminal 3 or the output device 29 via the interface 13. As a result, the occupational therapist or the user can obtain estimated data regarding the contents of the occupational therapy to be prescribed. The occupational therapist who has received the estimated data of prescription data directly or through the user can use the received estimated data for planning an occupational therapy plan and perform occupational therapy.

As described above, since the sleep data and the basic data of the subject 11 are objective data that do not depend on the evaluator, the estimated data computed by the artificial intelligence 23 on the basis of the sleep data and the basic data, that is, the estimated data regarding the activity of daily living data and the physical function data of the subject 11 are also objective data that do not depend on the evaluator. Similarly, since the estimated data regarding the activity of daily living data and the physical function data of the subject 11 are objective data that do not depend on the evaluator, the estimated data computed by the artificial intelligence 43 based on the estimated data, that is, the estimated data regarding the prescription data for the subject 11 are also objective data that do not depend on the evaluator. That is, according to the occupational therapy support device 102, the estimated data useful for the occupational therapy evaluation is obtained as the objective data independent of the evaluator regardless of whether or not the estimated data is output to the outside, and the estimated data useful for prescribing the occupational therapy is obtained as the objective data independent of the evaluator based on the estimated data. Note that the estimated data output unit 25 may output not only the estimated data regarding the prescription data of the subject 11 computed by the artificial intelligence 43 but also the estimated data computed by the artificial intelligence 23, that is, the estimated data regarding the activity of daily living data and the physical function data of the subject 11 to the outside via the interface 13, similarly to the occupational therapy support device 101.

Similarly to the artificial intelligence 23, the artificial intelligence 43 can output estimated data with high accuracy via machine learning. The occupational therapy support device 102 includes the input data reception unit 16, the training data reception unit 18, and the training unit 41, so that the occupational therapy support device 102 itself can train the artificial intelligence 43 without using an external artificial intelligence training device. That is, the occupational therapy support device 102 also incorporates an artificial intelligence training device that trains the artificial intelligence 43 as machine learning. When the occupational therapy support device 102 trains the artificial intelligence 43, the input data reception unit 16 receives the input of the input data including the activity of daily living data and the physical function data, and the training data reception unit 18 receives the input of the training data which is the correct prescription data corresponding to these input data. The training unit 41 inputs the input data whose input has been received by the input data reception unit 16 and the training data received by the training data reception unit 18 to the artificial intelligence 43 to train the artificial intelligence 43 so as to estimate the training data from the input data. Inputting a large number of sets of input data and training data associated with each other to the occupational therapy support device 102 will foster the training of the artificial intelligence 43 and improve the estimation accuracy.

In the past, the activity of daily living data and the physical function data collected for various subjects 11 and the prescription data indicating the prescription of the proven occupational therapy performed in correspondence with these data or the correct prescription to be performed are recorded in the storage medium 31 in association with each other, for example, so that the input data reception unit 16 and the training data reception unit 18 sequentially read a large number of data required for training from the storage medium 31, and the training unit 41 can repeat the training of the artificial intelligence 43 for each read data. As described above, the occupational therapy support device 102 can switch and execute two operation modes, namely, an estimation mode for computing and outputting estimated data using the artificial intelligence 43 and a training mode for training the artificial intelligence 43 as machine learning. For example, the input device 27 can instruct the switching of the operation modes. In the example of FIG. 5, the artificial intelligence 43 is incorporated in the computer 10 as part of the occupational therapy support device 102. On the other hand, as illustrated by the dotted line in FIG. 5, the artificial intelligence constructed in the external server 45 or the like may be used. In this case, the estimation unit 39, the training unit 41, and the estimated data output unit 25 operate the external artificial intelligence via the network 5 and the like. When external artificial intelligence is used, the artificial intelligence 43 constituting a part of the occupational therapy support device 102 is unnecessary.

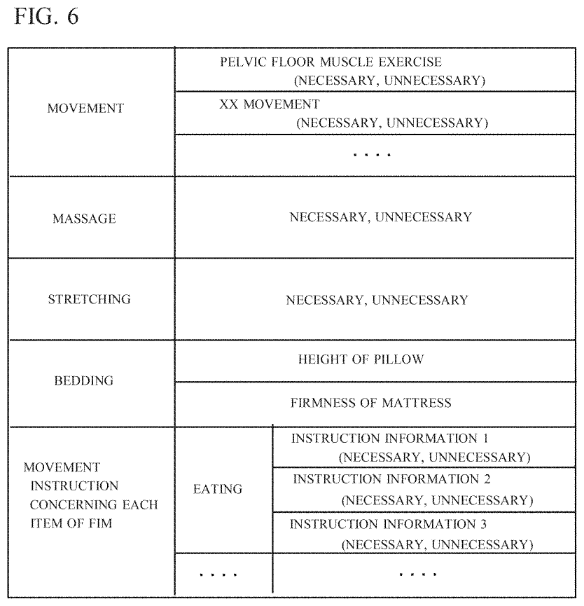

FIG. 6 is a tabular diagram illustrating prescription data that is output data of the occupational therapy support device 102. In the illustrated example, the prescription data includes prescription information for movement, massage, stretching, bedding conditions, and movement instruction information for each item of FIM. Regarding movement, for example, the prescription data includes various types of movement items in addition to pelvic floor muscle exercise, and the prescription data expresses whether or not the movement of each item is necessary. For example, when there are five types of movement items, prescription data regard movement is configured by a set of data indicating necessity such as (necessary, necessary, unnecessary, unnecessary, unnecessary). “Necessary” and “unnecessity” are represented by, for example, numerical values such as “1” and “0”. The prescription data regarding massage represents whether or not massage is necessary. The prescription data regarding stretching likewise represents whether stretching is necessary. The prescription data for bedding is, in the illustrated example, the height of a pillow and the firmness of a mattress. Each prescription data is expressed by, for example, a numerical value based on a predetermined unit. In addition to “necessary” and “unnecessary”, the information regarding movement, massage, and stretching may include body parts used for movement and the like, time, the number of times, necessity of a break to be taken between movements or the like, and a break time.

Regarding the movement instruction for each item of FIM, several pieces of instruction information are prepared in advance for each item. For example, if three types of instruction information 1, 2, and 3 are prepared for each item, prescription data containing movement instructions for all the 18 items of FIM is constituted by a set of 18 pieces of instruction information such as (2, 1, 3, . . . , 2). Each instruction information is represented by, for example, a numerical value such as “1”, “2”, or “3”. Alternatively, for example, “necessary” or “unnecessary” may be selected for each of the three types of movement information for each item of FIM. For example, selecting movement instructions such as (necessary, necessary, unnecessary) constitute prescription data containing a movement instruction for one item of FIM. Arranging such 18 prescription data will constitute prescription data containing movement instructions for all the 18 items of FIM. “Necessary” and “unnecessity” are represented by, for example, numerical values such as “1” and “0”. The estimated data output unit 25 converts the estimated value computed by the artificial intelligence 43, regarding the items of prescription data which are expressed by discontinuous numerical values such as “0”, “1”, “2”, . . . into numerical values each closest to one of the discontinuous numerical values by, for example, rounding off and outputs the converted values. When estimated data of (necessary, necessary, unnecessary) is obtained for a plurality of items that are not compatible with each other and should be selected alone, it is also possible to select an item closest to the code of “necessary”, for example, “1”. As an example of instruction information regarding the item “eating” illustrated in FIG. 6, the following are assumed: “suppress water intake” as instruction information 1 and “change drink from green tea to water” as instruction information 2. In these examples, when the sleep data indicates that the number of times of going to the bathroom is large, it is predicted that there is a high possibility of being “necessary”.

The estimated data of the prescription data output by the occupational therapy support device 102 may not include all the items exemplified in FIG. 6 but may include only some of them, for example, only one item. Even with only some items, the estimated data of obtained prescription data can be useful for prescribing occupational therapy. If the estimated data of the prescription data output by the occupational therapy support device 102 is sufficient to include only some items, it is sufficient to use only some items as training data also in the training of the artificial intelligence 43.

The description referring to FIGS. 3A to 3D and 6 has exemplified the form in which each item included in each of the sleep data, the basic data, the activity of daily living data, the physical function data, and the prescription data is expressed by a code associated in advance such as a numerical value. Code representation is convenient for the artificial intelligences 23 and 43 to handle. When each data is output to the output device 29 such as a display, it is desirable that each data is output not in the form of a message that can be read by the user, the occupational therapist, or the like, such as a sentence instead of a code without any change. Obviously, a code can be easily converted into a message form by an application of the user communication terminal 3 or the estimated data output unit 25 of the occupational therapy support device 101 or 102. Even if the data is converted into such a message form, the data is still estimated data.

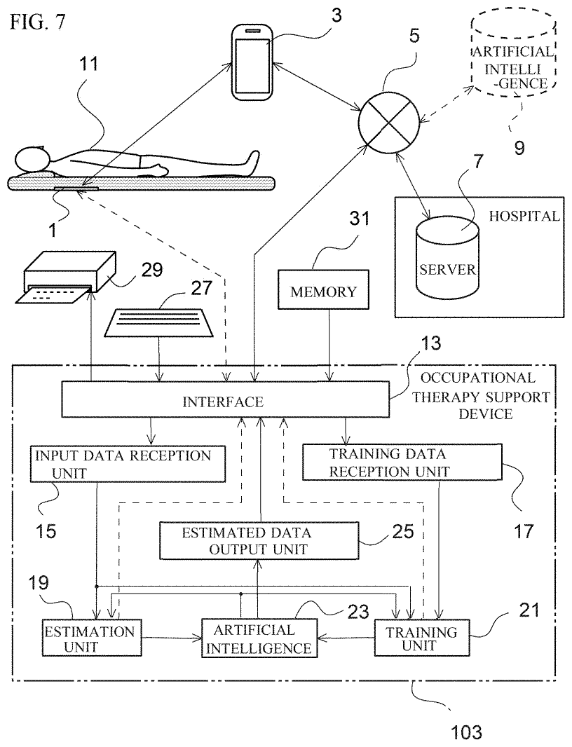

FIG. 7 is a block diagram illustrating the configuration of an occupational therapy support device according to still another embodiment of the present invention. An occupational therapy support device 103 is different from the occupational therapy support device 101 illustrated in FIG. 2 in that the estimation unit 19 and the artificial intelligence 23 add the estimated data output by the artificial intelligence 23, i.e., the past estimated data, to the new input data to the artificial intelligence 23. Thereby, the estimation accuracy of the artificial intelligence 23 can be improved.

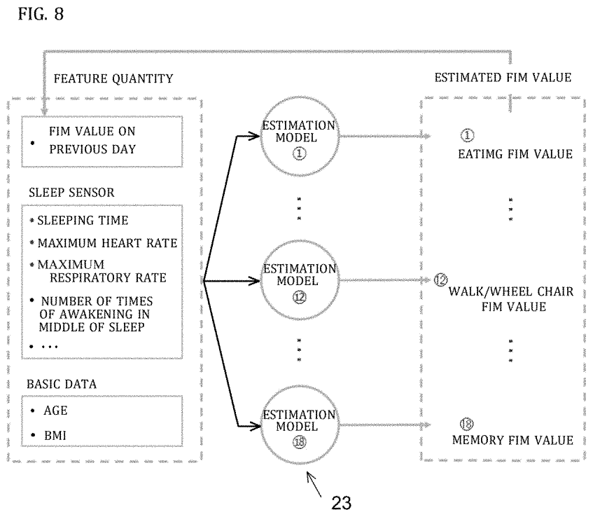

FIG. 8 is a block diagram showing input data and output data used in a verification test of the occupational therapy support device 103. The verification test supplied the occupational therapy support device 103 with a large number of combinations of the input data and the training data to thereby train the artificial intelligence 23, and evaluated the accuracy of the estimation performed by the artificial intelligence 23. As illustrated in the figure, in the verification test, the estimated FIM value on the previous day, which is the output data from the artificial intelligence 23, was added to the new input data.

The sleep sensor 1 used in the verification test was a commercially available one, and acquires data from a built-in pressure sensor at a sampling period of 16 Hz. The sleep sensor 1, based on a plurality of the acquired sampling data and through a software processing, computes and outputs data as measured data every minute, where the data are a respiratory rate (times/min), a heart rate (times/min), an amount of physical activity (body movement detection rate) (counts/min), the number of respiratory events (times/min), the number of detected convulsions (times/min), and a determination of leaving bed, lying on bed, and a sleeping state for every minute. The “amount of physical activity” means a frequency or intensity of body movements (movements of the body larger than respiration and heartbeat), and was only focused on the “frequency” in the verification test. The “respiratory event” means an apnea or hypopnea. The “sleeping state” in the “determination of leaving bed, lying on bed, and a sleeping state” means not only being in a state of lying on bed but also being in a state of sleeping. The data of the determination of leaving bed, lying on bed, and a sleeping state for every minute are each represented by a flag having a value of “0” or “1.”

For those measured data output by the sleep sensor 1, a maximum value, a minimum value, an average value and a dispersion value for every 3 hours are calculated and input to the input data reception unit 15 (see FIG. 7) as the input data. From the data of the determination of leaving bed, lying on bed, and a sleeping state for every minute among the measured data, sleeping time, non-sleeping time while lying, out-of-bed time, lying time, sleeping time/lying time, the number of times of leaving bed, the number of times of awakening in a middle of sleep for every 3 hours are calculated and input to the input data reception unit 15 (see FIG. 7) as the input data. Further, from the heart rate (times/min) among the measured data, in relation to the heart rate, a presence or absence of fluctuations not less than 10 bpm/min, an average during sleep—an average during non-sleep, an average during a recommended sleep period, a maximum during the recommended sleep period—an average during the recommended sleep period are calculated every 3 hours and input to the input data reception unit 15 (see FIG. 7) as the input data. In addition, from the respiratory rate (times/min) among the measured data, in relation to the respiratory rate, an average during sleep—an average during non-sleep, an average during a recommended sleep period, a maximum during the recommended sleep period—an average during the recommended sleep period are calculated every 3 hours and input to the input data reception unit 15 (see FIG. 7) as the input data.

“The number of times of leaving bed” means the number of times of moving from a lying position to leaving bed. “The number of times of awakening in a middle of sleep” means the number of times of awakening from sleeping in a lying position. “The presence or absence of fluctuations not less than 10 bpm/min” means whether there are fluctuations of 10 beats or more in a heart rate for one minute. For example, if the heart rate is 60 beats for one minute and 70 beats for the next one minute, it means that there has been a fluctuation of 10 bpm, and “the presence or absence of fluctuations not less than 10 bpm/min” is determined as “presence.” “The presence or absence” was represented by a flag having a value “1” or “0” as an example. Moreover, the determination of “the presence or absence” was performed by determining for every 3 hours whether there was “the fluctuations not less than 10 bpm/min” during the 3 hours.

In relation to the heart rate and the respiratory rate, the “average during sleep” and the “average during non-sleep” are averages for every 3 hours in a lying position. The “recommended sleep period” is a predetermined sleep time zone, and, in the verification test, was set as a time zone from 9:00 pm to 6:00 am on the next day, which is common as a lights-out period in hospitals. Hence, the “average during a recommended sleep period” and the “maximum during the recommended sleep period” respectively mean an average and a maximum for every 3 hours in the “recommended sleep period” (i.e., for each of periods from 9:00 pm to 0:00 am on the next day, from 0:00 am to 3:00 am, and from 3:00 am to 6:00 am).

It should be noted that, in the verification test, the data for one day were input at one time as the input data and the range of one day was set as a period from 6:00 am to 6:00 am on the next day.

In the verification test, the number of samples used for the machine learning is 325 person-days. As the artificial intelligence 23 used in the verification test, a decision tree-based LGBM (Light GBM; manufactured by Microsoft Corporation) was used. Since the LGBM advantageously enables a user to easily analyze which variables play an important role on the estimated value contrary to the neural network illustrated in FIG. 4, the LGBM seemed to be especially useful for the verification test with a view to leading to future improvements.

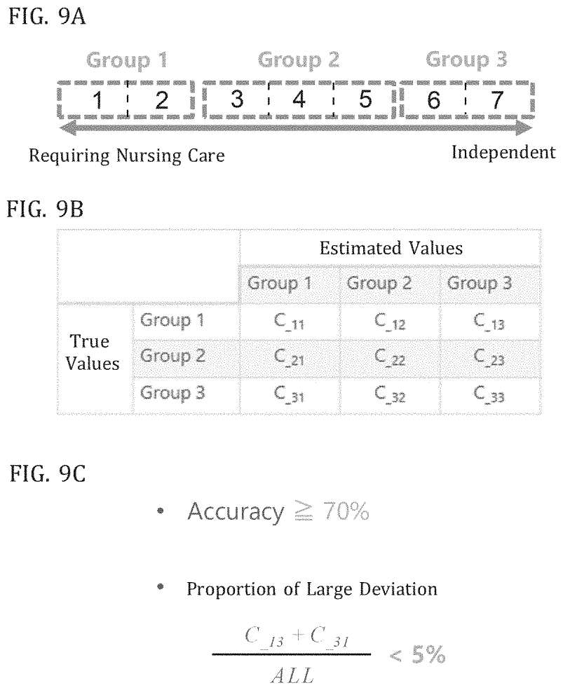

FIGS. 9A to 9C are explanatory diagrams showing an evaluation method used in the verification test of the occupational therapy support device 103. Since the number of samples was limited in the verification test, the 7-level evaluation values for each FIM item were replaced with 3-level evaluation values corresponding to groups 1 to 3 as shown in FIG. 9A. As shown in FIG. 9B, for the evaluation values for each FIM item, the relationship between the true values as the training data and the estimated values as the output data can be expressed by a 3-row and 3-column matrix. Each matrix element Cij represents the proportion of the number of samples having the corresponding relationship.

As shown in FIG. 9C, the estimation accuracy (Accuracy) being 70% or more and the proportion of large deviation being less than 5% was determined as criteria for a practical level. The estimation accuracy corresponds to the proportion of the diagonal elements of the matrix in FIG. 9B to the whole. The large deviation corresponds to either a case where a sample belonging to group 1 is estimated to be in group 3 or a case where a sample belonging to group 3 is estimated to be in group 1. Therefore, the proportion of the large deviation is represented by the proportion of the sum of component C13 and component C31 to the total.

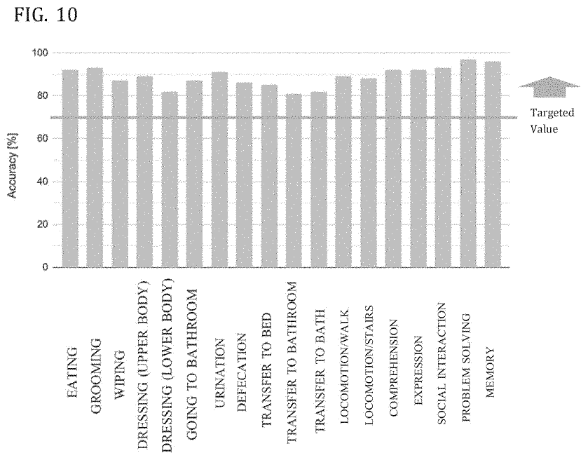

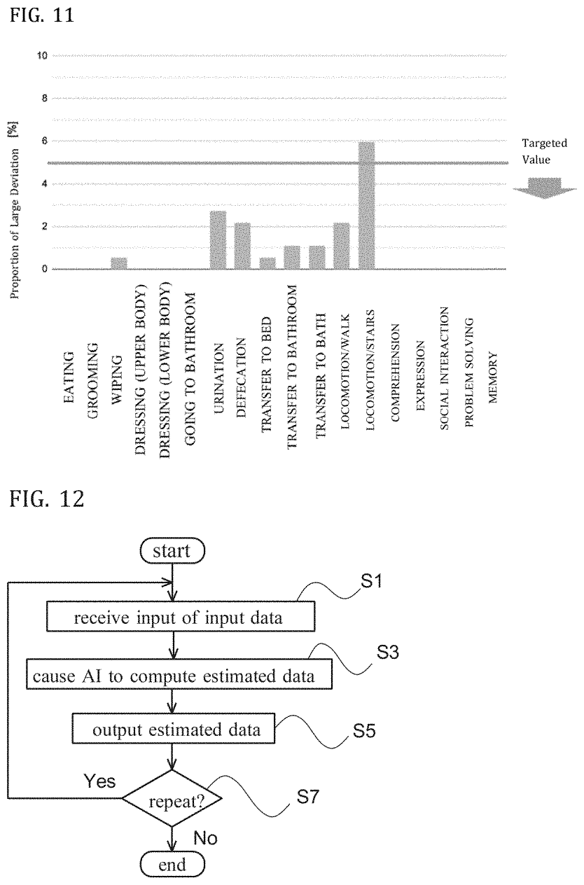

FIG. 10 and FIG. 11 are graphs showing the results of the demonstration tests of the occupational therapy support device 103. In the demonstration test, cross-validation (Leave one subject) was used to evaluate accuracy. As the graph in FIG. 10 shows, the estimation accuracy exceeded the targeted 70% and even exceeded 80% for all 18 FIM items. As shown in the graph in FIG. 11, for all ones among the 18 FIM items excluding “stairs”, the proportion of large deviation was far below the targeted criteria 5% and was less than 3%. As for the “stairs”, it is possible to improve the accuracy by eliminating data bias. Thus, it was demonstrated that even though the number of samples for machine learning was limited, a practical level of estimation accuracy could be obtained.

### OTHER EMBODIMENTS

In the above, as the occupational therapy support devices 101, 102, and 103, examples in which the input data includes the basic data (see FIG. 3B) have been shown. On the other hand, the present invention can also be implemented in a form in which the input data does not contain the basic data. Even in such a form, reasonably high-accuracy estimation data can be obtained.

As the sleep data, it is possible to select any one of information on pulse, information on respiration, and information on body movements, or to select at least one of them. In this way, it is also possible to compute the estimated data of activities of daily living based on a minimum number of items among the sleep data of subjects for occupational therapy evaluation, and reasonably high-accuracy estimation data can be obtained.

Instead of the estimated data output by the artificial intelligence 23, that is, the past estimated data illustrated in the occupational therapy support device 103, past FIM values that are not estimated data may be added to the new input data of the artificial intelligence 23. This form also improves the accuracy of the estimated data.

The form of adding the estimated data output by the artificial intelligence 23 or the past FIM values that are not the estimated data to the new input data of the artificial intelligence 23 can also be applied to the occupational therapy support device 102.

In the above, an example of adopting the evaluation data of the evaluation items defined in the Functional Independence Measure (FIM) as the activity of daily living data was shown. On the other hand, as the activity of daily living data, it is possible to adopt not only FIM but also evaluation data of other evaluation items related to the activity of daily living.

Any one of the occupational therapy support devices 101 (FIG. 2), 102 (FIG. 5), and 103 (FIG. 7) implements the occupational therapy support method and the artificial intelligence training method. FIGS. 12 to 14 illustrate the processing procedures of the occupational therapy support method and the artificial intelligence training method.

FIG. 12 is a flow chart illustrating the process flow of the occupational therapy support method implemented by the occupational therapy support devices 101 (FIG. 2) and 103 (FIG. 7). When the process is started, the input data reception unit 15 receives input of input data (S1). Next, the estimation unit 19 inputs the input data to the artificial intelligence 23 that has already been trained, thereby causing the artificial intelligence 23 to compute estimated data (S3). In the case of the occupational therapy support device 103, the estimation unit 19 adds past estimated data having been estimated by the artificial intelligence 23 to the input data to be input to the artificial intelligence 23. Next, the estimated data output unit 25 outputs the estimated data computed by the artificial intelligence 23 (S5). Next, the occupational therapy support devices 101 and 103 return the process to S1 when the process should be repeated based on the user's instruction or the like (Yes in S7). Thereby, the input data reception unit 15 receives input of new input data. The occupational therapy support devices 101 and 103 terminate the process when the process should not be repeated (No in S7).

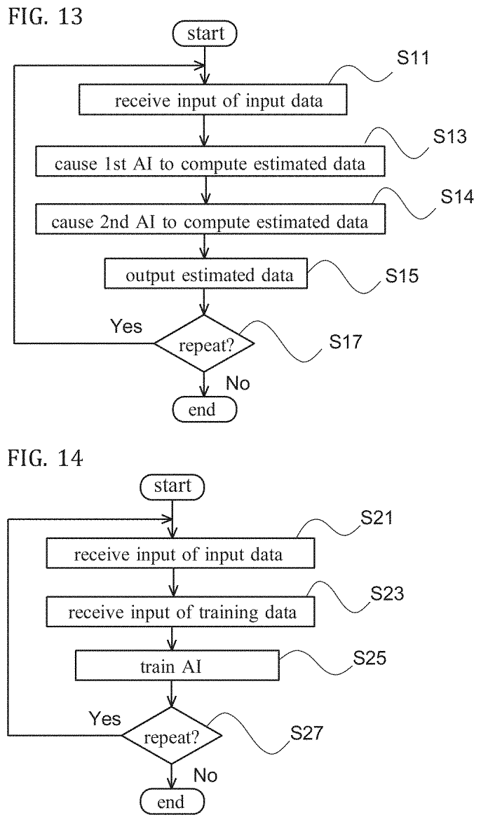

FIG. 13 is a flow chart illustrating the process flow of the occupational therapy support method implemented by the occupational therapy support device 102 (FIG. 5). When the process is started, the input data reception unit 15 receives input of input data (S11). Next, the estimation unit 19 inputs the input data to the first artificial intelligence 23 that has already been trained, and causes the first artificial intelligence 23 to compute estimated data (S13). As with the occupational therapy support device 103, the estimation unit 19 may add past estimated data having been estimated by the artificial intelligence 23 to the input data to be input to the artificial intelligence 23. Next, the estimation unit 39 inputs the estimated data computed by the first artificial intelligence 23 to the second artificial intelligence 43 that has already been trained, thereby causing the second artificial intelligence 43 to compute estimated data (S14). Next, the estimated data output unit 25 outputs the estimated data computed by the second artificial intelligence 43 (S15). Next, the occupational therapy support device 102 returns the process to S11 when the process should be repeated based on the user's instruction or the like (Yes in S17). Thereby, the input data reception unit 15 receives input of new input data. The occupational therapy support device 102 terminates the process when the process should not be repeated (No in S17).

FIG. 14 is a flow chart illustrating the processing flow of the artificial intelligence training method implemented by the occupational therapy support devices 101 (FIG. 2), 102 (FIG. 5), and 103 (FIG. 7). When the process is started, the input data reception units 15 and 16 receive input of input data (S21). The training data reception units 17 and 18 receive input of training data (S23). Either the process S21 or the process S23 may be performed first, or may be performed at the same time. Next, the training units 21 and 41 input the input data and the training data to the artificial intelligences 23 and 43 to train the artificial intelligences to estimate the training data from the input data (S25). In the case of the occupational therapy support device 103, the training unit 21 adds past estimated data having been estimated by the artificial intelligence 23 to the input data to be input to the artificial intelligence 23. Similarly, in the occupational therapy support device 102, the training unit 21 may add past estimated data having been estimated by the artificial intelligence 23 to the input data to be input to the artificial intelligence 23.

Next, the occupational therapy support devices 101, 102, and 103 return the process to S21 when the process should be repeated based on the user's instruction or the like (Yes in S27). As a result, the input data reception units 15 and 16 receive new input data, and the training data reception units 17 and 18 receive new training data. The occupational therapy support devices 101, 102, 103 terminate the process when the process should not be repeated (No in S27).

As already mentioned, the occupational therapy support device 101 (see FIG. 2) is incorporated in the computer 10 in the example shown in FIG. 1. The computer 10 functions as the occupational therapy support device 101 by a specific application, that is, program, being installed in the computer 10 and started. This program may be supplied through the network 5, or may be supplied by the storage medium 31 (see FIG. 2) such as a CDROM. The same applies to the occupational therapy support devices 102 (see FIG. 5) and 103 (see FIG. 7).

In the above description, an occupational therapist has been described as an example of a person who uses the estimated data output from the occupational therapy support devices 101, 102 and 103. However, a person who performs occupational therapy using estimated data is not limited to an occupational therapist and may be, for example, a user including the subject 11 or may be another medical or care worker such as a doctor, a physical therapist, or a care worker. In particular, there is an advantage that the estimated data of the prescription data output by the occupational therapy support device 102 is easily used by general users.

This application is based on Japanese Patent Application No. 2021-098380 filed by the applicant and its co-applicant in Japan on Jun. 13, 2021, the entire contents of which are incorporated herein by reference.

The above description of specific embodiments of the present invention has been presented for the purpose of illustration. They are not intended to be exhaustive or to limit the invention as it is in the form described. It is obvious to those skilled in the art that many modifications and variations are possible in light of the above description.

### REFERENCE SIGNS LIST

1 sleep sensor
3 user communication terminal
5 network
7, 9 server
10 computer
11 subject
13 interface
15, 16 input data reception unit
17, 18 training data reception unit
19 estimation unit
21 training unit
23 artificial intelligence
25 estimated data output unit
27 input device
29 output device
31 memory
33 input layer
35 intermediate layer
37 output layer
39 estimation unit
41 training unit
43 artificial intelligence
100 occupational therapy assistance system
101, 102 occupational therapy support device.

1. An occupational therapy support device comprising:
a processor that executes instructions to:
receive input data including sleep data regarding sleep of a subject of occupational therapy evaluation;
input the input data received to a trained artificial intelligence to cause the artificial intelligence to compute estimated data of data including activity of daily living data regarding activity of daily living of the subject; and
output the estimated data computed by the artificial intelligence, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

2. An occupational therapy support device comprising:
a processor that executes instructions to:
receive input data including sleep data regarding sleep of a subject of occupational therapy evaluation;
input the input data received to a trained first artificial intelligence to cause the first artificial intelligence to compute estimated data of data including activity of daily living data regarding activity of daily living of the subject;
input the estimated data computed by the first artificial intelligence to a trained second artificial intelligence to cause the second artificial intelligence to compute estimated data of prescription data of occupational therapy for the subject; and
output the estimated data of the prescription data computed by the second artificial intelligence,
wherein the prescription data includes data expressing contents to be prescribed for at least one item of movement, massage, stretching, and a bedding condition,
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

3-13. (canceled)

14. An artificial intelligence training device for occupational therapy support, the artificial intelligence training device being for training an artificial intelligence to be used for the occupational therapy support, the artificial intelligence training device comprising:
a processor that executes instructions to:
receive input data including sleep data regarding sleep of a subject of occupational therapy evaluation;
receive training data including activity of daily living data regarding activity of daily living of the subject, the training data corresponding to the input data; and
input the input data received and the training data received to an artificial intelligence to train the artificial intelligence so as to estimate the training data from the input data, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

15-24. (canceled)

25. An artificial intelligence training device for occupational therapy support, the artificial intelligence training device being for training an artificial intelligence to be used for the occupational therapy support, the artificial intelligence training device comprising:
a processor that executes instructions to:
receive input data including activity of daily living data regarding activity of daily living of a subject of occupational therapy evaluation;
receive training data that is prescription data of occupational therapy for the subject, the training data corresponding to the input data; and
input the input data received and the training data received to an artificial intelligence to train the artificial intelligence so as to estimate the training data from the input data, wherein
the prescription data includes data expressing contents to be prescribed, regarding at least one item of movement, massage, stretching, and a bedding condition, and
the activity of daily living data is data of an item regarding activity of daily living and including data evaluated in plural levels on at least one of evaluation items defined in Functional Independence Measure.

26-45. (canceled)

46. An occupational therapy support method comprising:
inputting input data including sleep data regarding sleep of a subject of occupational therapy evaluation to a trained artificial intelligence to cause the artificial intelligence to compute estimated data of data including activity of daily living data regarding activity of daily living of the subject;
outputting the estimated data computed by the artificial intelligence; and
performing occupational therapy to the subject in accordance with the estimated data output, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

47. An occupational therapy support method comprising:
inputting input data including sleep data regarding sleep of a subject of occupational therapy evaluation to a trained first artificial intelligence to cause the first artificial intelligence to compute estimated data of data including activity of daily living data regarding activity of daily living of the subject;
inputting the estimated data computed by the first artificial intelligence to a trained second artificial intelligence to cause the second artificial intelligence to compute estimated data of prescription data of occupational therapy for the subject;
outputting the estimated data of the prescription data computed by the second artificial intelligence; and
performing occupational therapy to the subject in accordance with the estimated data output, wherein
the prescription data includes data expressing contents to be prescribed for at least one item of movement, massage, stretching, and a bedding condition,
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

48. An artificial intelligence training method for occupational therapy support, the artificial intelligence training device being for training an artificial intelligence to be used for the occupational therapy support, the artificial intelligence training method comprising:
preparing data sets of input data including sleep data regarding sleep of a subject of occupational therapy evaluation, and corresponding training data including activity of daily living data regarding activity of daily living of the subject: and
inputting the prepared data sets to an artificial intelligence to train the artificial intelligence so as to estimate the training data from the input data, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

49. An artificial intelligence training method for occupational therapy support, the artificial intelligence training device being for training an artificial intelligence to be used for the occupational therapy support, the artificial intelligence training method comprising:
preparing data sets of input data including activity of daily living data regarding activity of daily living of a subject of occupational therapy evaluation, and corresponding training data that is prescription data of occupational therapy for the subject; and
inputting the prepared data sets to an artificial intelligence to train the artificial intelligence so as to estimate the training data from the input data, wherein
the prescription data includes data expressing contents to be prescribed, regarding at least one item of movement, massage, stretching, and a bedding condition, and
the activity of daily living data is data of an item regarding activity of daily living and including data evaluated in plural levels on at least one of evaluation items defined in Functional Independence Measure.

50. A non-transitory computer-readable medium storing an occupational therapy support program including instructions that, when executed by a processor, causes a computer, to:
receive input data including sleep data regarding sleep of a subject of occupational therapy evaluation;
input the input data received to a trained artificial intelligence to cause the artificial intelligence to compute estimated data of data including activity of daily living data regarding activity of daily living of the subject; and
output the estimated data computed by the artificial intelligence, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

51. A non-transitory computer-readable medium storing an occupational therapy support program including instructions that, when executed by a processor, causes a computer, to:
receive input data including sleep data regarding sleep of a subject of occupational therapy evaluation;
input the input data received to a trained first artificial intelligence to cause the first artificial intelligence to compute estimated data of data including activity of daily living data regarding activity of daily living of the subject;
input the estimated data computed by the first artificial intelligence to a trained second artificial intelligence to cause the second artificial intelligence to compute estimated data of prescription data of occupational therapy for the subject; and
output the estimated data of the prescription data computed by the second artificial intelligence,
wherein the prescription data includes data expressing contents to be prescribed for at least one item of movement, massage, stretching, and a bedding condition,
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

52. A non-transitory computer-readable medium storing an artificial intelligence training program, the program including instructions that, when executed by a processor, causes a computer, to:
receive input data including sleep data regarding sleep of a subject of occupational therapy evaluation;
receive training data including activity of daily living data regarding activity of daily living of the subject, the training data corresponding to the input data; and
input the input data received and the training data received to an artificial intelligence to train the artificial intelligence so as to estimate the training data from the input data, wherein
the sleep data includes data that is based on data measured by a sleep sensor,
the activity of daily living data is data of an item regarding activity of daily living and including data evaluated in plural levels on at least one of evaluation items defined in Functional Independence Measure, and
the data that is based on data measured by the sleep sensor includes at least one data item selected from the group consisting of sleeping time, non-sleeping time while lying, out-of-bed time, lying time, sleeping time/lying time, the number of times of leaving bed, the number of times of awakening in a middle of sleep, presence or absence of fluctuations in heart rate not less than a predetermined level, an average heart rate during sleep—an average heart rate during non-sleep, an average heart rate during a predetermined sleep period, a maximum heart rate during the predetermined sleep period—an average heart rate during the predetermined sleep period, an average respiratory rate during sleep—an average respiratory rate during non-sleep, an average respiratory rate during the predetermined sleep period, and a maximum respiratory rate during the predetermined sleep period—an average respiratory rate during the predetermined sleep period, wherein each data item of the group is for every predetermined time.

53. A non-transitory computer-readable medium storing an artificial intelligence training program, the program including instructions that, when executed by a processor, causes a computer, to:
receive input data including activity of daily living data regarding activity of daily living of a subject of occupational therapy evaluation;
receive training data that is prescription data of occupational therapy for the subject, the training data corresponding to the input data; and
input the input data received and the training data received to an artificial intelligence to train the artificial intelligence so as to estimate the training data from the input data, wherein
the prescription data includes data expressing contents to be prescribed, regarding at least one item of movement, massage, stretching, and a bedding condition, and
the activity of daily living data is data of an item regarding activity of daily living and including data evaluated in plural levels on at least one of evaluation items defined in Functional Independence Measure.
